(19) United States
(12) Patent Application Publication
(10) Pub. No.: US 2005/0260179 A1
(43) Pub. Date: Nov. 24, 2005

(54) Stem and progenitor cell capture for tissue regeneration

(75) Inventors: Mark C. Fishman, Newton Center, MA (US); Linda G. Griffith, Cambridge, MA (US); Paul Huang, Boston, MA (US)

Correspondence Address: PATREA L. PABST;PABST PATENT GROUP LLP, 400 COLONY SQUARE, SUITE 1200, ATLANTA, GA, 30361, US

(73) Assignees: The General Hospital Corporation D/B/A Massachusetts General Hospital, Boston, MA (US); Massachusetts Institute of Technology, Cambridge, MA (US)

(21) Appl. No.: 10/494,817

(22) PCT Filed: Nov. 6, 2002
(86) PCT No.: PCT/US02/35699

§ 371 Date: Sep. 20, 2004

Related U.S. Application Data
(60) Provisional application No. 60/332,935, filed on Nov. 6, 2001.

(51) Int. Cl. A61K 48/00; C12N 5/08

(52) U.S. Cl. 424/93.21; 435/366

(57) ABSTRACT

A method for capturing stem cells and/or progenitor cells from circulating body fluids such as blood or lymph in vivo and enabling the cells to colonize a specific, localized site for purposes of tissue regeneration or repair, has been developed. The method consists of selecting a site in need of repair or regeneration. Materials which can be used to provide the growth factors or ligands include polymeric matrices having the growth factor or ligands attached thereto, materials releasing growth factor or ligands (such as hydrogels, polymers, fibrin clots), and cells which release or have bound to their surfaces the growth factors or ligands.

### BACKGROUND OF THE INVENTION

The present invention is generally in the field of tissue engineering, and specifically the use of immobilized ligands to capture stem and/or progenitor cells at a site where tissue regeneration is needed.

Tissue engineering has emerged as a scientific field having the potential to aid in human therapy by producing anatomic tissues and organs for the purpose of reconstructive surgery and transplantation. It combines the scientific fields of materials science, cell and molecular biology, and medicine to yield new devices for replacement, repair, and reconstruction of tissues and structures within the body. Many approaches have been advocated over the last decade. One approach is to combine tissue specific cells with open porous polymer scaffolds which can then be implanted. Large numbers of cells can be added to the polymer device in cell culture and maintained by diffusion. After implantation, vascular ingrowth occurs, the cells remodel, and a new stable tissue is formed as the polymer degrades by hydrolysis.

A number of approaches have been described for fabricating tissue regeneration devices for either in vitro or in vivo growth of cells. Polymeric devices have been described for replacing organ function or providing structural support. Such methods have been reported by Vacanti, et al., Arch. Surg. 123:545-49 (1988); U.S. Pat. No. 4,060,081 to Yannas, et al.; U.S. Pat. No. 4,485,097 to Bell; and U.S. Pat. No. 4,520,821 to Schmidt, et al. In general, the methods used by Vacanti, et al., and Schmidt, et al., can be practiced by selecting and adapting existing polymer fiber compositions for implantation and seeding with cells, while the methods of Yannas and Bell produce very specific modified collagen sponge-like structures.

Tissue regeneration devices must be porous with interconnected pores to allow cell and tissue penetration, if the device is of any significant thickness. Factors such as pore size, shape, and tortuosity can all affect tissue ingrowth but are difficult to control using standard processing techniques. U.S. Pat. No. 5,518,680 to Cima et al. describes the use of solid free form fabrication techniques, especially three dimensional printing of polymer powders, to form matrices which can be seeded with dissociated cells and implanted to form new structures. The advantages of the solid free form methods to construct specific structures from biocompatible synthetic or natural polymers, inorganic materials, or composites of inorganic materials with polymers, where the resulting structure has defined pore sizes, shapes and orientations, particularly different pore sizes and orientations within the same device, with more than one surface chemistry or texture at different specified sites within the device, is readily apparent. PCT/US96/09344 to Massachusetts Institute of Technology and Childrens' Medical Center Corporation describes the use of solid free-form fabrication (SFF) methods to manufacture devices for allowing tissue regeneration and for seeding and implanting cells to form organ and structural components, which can additionally provide controlled release of bioactive agents, wherein the matrix is characterized by a network of lumens functionally equivalent to the naturally occurring vasculature of the tissue formed by the implanted cells, and which can be lined with endothelial cells and coupled to blood vessels or other ducts at the time of implantation to form a vascular or ductile network throughout the matrix.

A major limitation on the use of these structures is the type of cell that can be cultured in vitro and seeded onto and into the matrix. It would be very desirable if one could put stem cells or early progenitor cells onto or into the matrix, prior to implantation, but this is difficult in view of the limited number of cells available. It would be even more desirable if one could implant a matrix or other material and induce stem or progenitor cells to migrate to the site, attach, preferably proliferate, and form new differentiated tissue at the site.

It is therefore an object of the present invention to provide methods and materials for inducing tissue healing and regeneration by stem and/or progenitor cells.

It is a further object of the present invention to provide a means for capturing stem cells or progenitor cells at a site where tissue regeneration is needed.

### SUMMARY OF THE INVENTION

Studies have now demonstrated that even for such differentiated tissue as heart muscle tissue, stem cells or early progenitor cells migrate through the body to areas of damage, such as infarcted heart tissue, and form new tissue. A method has now been developed whereby the circulating stem or progenitor cells can be captured at the site where tissue regeneration is needed. In the preferred embodiment, repair can be induced by introduction into the tissue to be repaired of a matrix to which cells attach, in some cases proliferate, and differentiate. In another embodiment, growth factors or other ligands are provided at the site, preferably released from a controlled or sustained release material or from cells which produce the growth factors or ligands.

### DETAILED DESCRIPTION OF THE INVENTION

A method for capturing stem cells and/or progenitor cells from circulating body fluids such as blood or lymph in vivo and enabling the cells to colonize a specific, localized site for purposes of tissue regeneration or repair, has been developed. The method consists of selecting a site in need of repair and providing at the site growth factors or ligands which selectively bind to stem cells or progenitor cells for the tissue in need of repair or regeneration. Materials which can be used to provide the growth factors or ligands include polymeric matrices having the growth factor or ligands attached thereto, materials releasing growth factor or ligands (such as hydrogels, polymers, fibrin clots), and cells which release or have bound to their surfaces the growth factors or ligands.

I. Cells to be Captured.

Any site in need of repair or regeneration can be treated using the methods described herein. The goal is to immobilize stem cells or progenitor cells which differentiate at the site to form a specific kind of tissue. A specific example is the selection of circulating hemangioblasts or angioblasts for repair of blood vessels, or for initiating angiogenesis in damaged cardiac muscle. Additional examples include selection of cardioblasts that can be induced to differentiate into cardiac muscle, and selection of circulating cells that can be induced under local signals to differentiate into liver and neural tissue.

II. Materials for Capture

Ligands

Ligands, as used herein, refer to molecules that bind to cell surface receptors and regulate the growth, replication or differentiation of target cells or tissue. Preferred ligands are growth factors and extracellular matrix molecules. Examples of growth factors include epidermal growth factor (EGF), platelet-derived growth factor (PDGF), transforming growth factors (TGFα, TGFβ), hepatocyte growth factor, heparin binding factor, insulin-like growth factor I or II, fibroblast growth factor, erythropoietin, nerve growth factor, bone morphogenic proteins, muscle morphogenic proteins, and other factors known to those of skill in the art. Additional growth factors are described in “Peptide Growth Factors and Their Receptors I” M. B. Sporn and A. B. Roberts, eds. (Springer-Verlag, New York, 1990), for example. Factors include vascular endothelial cell growth factor (VEGF), c-kit ligand (st1 factor), tenascin, fibroblast growth factor (FGF), ligands for alpha 4 beta 1 integrin, and cytokines such as leukemia inhibitory factor (LIF).

Growth factors can be isolated from tissue using methods know to those of skill in the art. For example, growth factors can be isolated from tissue, produced by recombinant means in bacteria, yeast or mammalian cells. For example, EGF can be isolated from the submaxillary glands of mice and Genentech produces TGF-β recombinantly. Many growth factors are also available commercially from vendors, such as Sigma Chemical Co. of St. Louis, Mo., Collaborative Research, Genzyme, Boehringer, R&D Systems, and GIBCO, in both natural and recombinant forms.

Examples of extracellular matrix molecules include fibronectin, laminin, collagens, and proteoglycans. Other extracellular matrix molecules are described in Kleinman et al. (1987) or are known to those skilled in the art. Other ligands useful for tethering include cytokines, such as the interleukins and GM-colony stimulating factor, and hormones, such as insulin. These are also described in the literature and are commercially available.

In the preferred embodiment, the ligand is specific for stem cells or progenitor cells that differentiate into the desired tissue. Many of the growth factors described above are fairly general in their specificity. Accordingly, it may be desirable to select more specific ligands, which may be antibodies to specific cell markers. Ligands may be selected to capture the stem or progenitor cells, to induce proliferation of the captured cells, or to induce differentiation. Ligands effective for achieving these targets are known to those skilled in the art.

Immobilization Means

The ligands can be administered directly to the site where they are needed, produced by cells administered directly to the site where they are needed, or released over a period of time from a matrix material or depo at the site where they are needed.

In the preferred embodiment, the ligands are tethered to or incorporated into a polymeric material. If tethered, the ligands are preferably bound via a tether that does not interfere with binding of the cells to the ligand. Alternatively, the ligand can be incorporated into a polymeric material using known techniques, so that it is released at the site where the cells are to be captured.

As used herein, a tether is a flexible link between an attachment matrix and a ligand. Flexible tethers for attaching ligands to a matrix must satisfy two important requirements: (1) the need for mobility of the ligand-receptor complex within the cell membrane in order for the ligand to exert an effect, and (2) biocompatibility of materials used for immobilization. Substantial mobility of a tethered growth factor is critical because even though the cell does not need to internalize the complex formed between the receptor and the growth factor, it is believed that several complexes must cluster together on the surface of the cell in order for the growth factor to stimulate cell growth. In order to allow this clustering to occur, the growth factors are attached to the solid surface, for example, via long water-soluble polymer chains, which are referred to as tethers, allowing movement of the receptor-ligand complex in the cell membrane.

Examples of water-soluble, biocompatible polymers which can serve as tethers include polymers such as synthetic polymers like polyethylene oxide (PEO), polyvinyl alcohol, polyhydroxyethyl methacrylate, polyacrylamide, and natural polymers such as hyaluronic acid, chondroitin sulfate, carboxymethylcellulose, and starch.

Tethers can also be branched to allow attachment of multiple ligands in close proximity. Branched tethers can be used, for example, to increase the density of ligand on the matrix. Such tethers are also useful in bringing multiple or different ligands into close proximity on the cell surface. This is useful when using a combination of different ligands. Preferred forms of branched tethers are star PEO and comb PEO.

Star PEO is formed of many PEO “arms” emanating from a common core. Star PEO has been synthesized, for example, by living anionic polymerization using divinylbenzene (DVB) cores, as described by Gnanou et al., Makromol. Chemie 189: 2885-2892 (1988), and Merrill, J. Biomater. Sci. Polymer Edn 5: 1-11 (1993). The resulting molecules have 10 to 200 arms, each with a molecular weight of 3,000 to 12,000. These molecules are about 97% PEO and 3% DVB by weight. Other core materials and methods may be used to synthesize star PEO. Comb PEO is formed of many PEO chains attached to and extending from the backbone of another polymer, such as polyvinyl alcohol. Star and comb polymers have the useful feature of grouping together many chains of PEO in close proximity to each other.

Length

The length of a tether is limited only by the mechanical strength of the tether used and the desired stability of a tethered growth factor. It is expected that stronger tethers can be made longer than weaker tethers, for example. It is also desirable for tether length and strength to be matched to give a desired half life to the tether, prior to breakage, and thereby adjust the half life of growth factor action. The minimum tether length also depends on the nature of the tether. A more flexible tether will function well even if the tether length is relatively short, while a stiffer tether may need to be longer to allow effective contact between a cell and the ligands.

The backbone length of a tether refers to the number of atoms in a continuous covalent chain from the attachment point on the matrix to the attachment point of the ligand. All of the tethers attached to a given matrix need not have the same backbone length. In fact, using tethers with different backbone lengths on the same matrix can make the resulting composition more effective and more versatile. In the case of branched tethers, there can be multiple backbone lengths depending on where and how many ligands are attached. Preferably, tethers can have any backbone length between 5 and 50,000 atoms. Within this preferred range, it is contemplated that backbone length ranges with different lower limits, such as 10, 15, 25, 30, 50, and 100, will have useful characteristics.

Such tethers are not intended to be limited by the manner in which the matrix-tether-ligand composition is assembled. For example, if linker molecules are attached to the matrix and the ligand, and then the linkers are joined to form the tethered composition, the entire length of the joined linkers is considered the tether. As another example, the attachment matrix may, by its nature, have on its surface protruding molecular chains. If a linker molecule is attached to the matrix via such protruding chains, then the chain and linker together are considered to be a tether.

Biocompatible polymers and spacer molecules are well known in the art and most are expected to be suitable for forming tethers. The only important characteristics are biocompatibility and flexibility. That is, the tether should not be made of a substance that is cytotoxic or, in the case of in vivo uses, which causes significant allergic or other physiological reaction when implanted. The tether should also allow the growth factor a sufficient range of motion to effectively bind to a cell surface receptor.

The biodegradability of a tether, the tether-matrix link, or the tether-growth factor link can be used to regulate the length of time a growth factor stimulates growth. For example, if a given tether degrades during cell growth at a consistent rate, then a limit can be placed on how long the growth factors binds to and stimulates cell growth. Once untethered, a growth factor can be internalized by the cell or can diffuse away from the target cells. Such planned degradation is especially useful in the context of implanted compositions, used to stimulate tissue replacement, by limiting the amount of tissue growth.

Matrix Materials

There are two basic types of substrates onto which ligands can be tethered. One class includes biocompatible materials which are not biodegradable, such as polystyrenes, polyethylene vinyl acetates, polypropylenes, polymethacrylates, polyacrylates, polyethylenes, polyethylene oxides, glass, polysilicates, polycarbonates, polytetrafluoroethylene, fluorocarbons, nylon, silicon rubber, and stainless steel alloys. The other class of materials includes biocompatible, biodegradable materials such as polyanhydrides, polyglycolic acid, polyhydroxy acids such as polylactic acid, polyglycolic acid, and polylactic acid-glycolic acid copolymers, polyorthoesters, polyhydroxybutyrate, polyphosphazenes, polypropylfumerate, and biodegradable polyurethanes, proteins such as collagen and polyamino acids, and polysaccharides such as glycosaminoglycans, alginate, and carageenan, bone powder or hydroxyapatite, and combinations thereof. These biodegradable polymers are preferred for in vivo tissue growth scaffolds. Other degradable polymers are described by Engleberg and Kohn, Biomaterials 12: 292-304 (1991).

Attachment substrates can have any useful form including fibers, woven fibers, shaped polymers, particles and microparticles. Woven fibers are useful for stimulating growth of tissue in the form of a sheet, sponge or membrane.

The biodegradability of a matrix can be used to regulate the length of time the growth factor stimulates growth and to allow replacement of implanted matrix with new tissue. For this purpose the matrix with tethered ligands can be considered a scaffold upon which new tissue can form. As such, a degradable scaffold is broken down as tissue replacement proceeds. Once released from the matrix, a growth factor can be internalized or can diffuse away from the target cells. Such planned degradation is especially useful in the context of implanted compositions, used to stimulate tissue replacement, by limiting the amount of tissue growth and eliminating the need to remove the tissue scaffold. For implantation in the body, preferred degradation times are typically less than one year, more typically in the range of weeks to months.

In some embodiments, attachment of the cells to the matrix is enhanced by coating the matrix with compounds such as extracellular membrane components, basement membrane components, agar, agarose, gelatin, gum arabic, collagen types I, II, III, IV, and V, fibronectin, laminin, glycosaminoglycans, mixtures thereof, and other materials known to those skilled in the art of cell culture.

A non-thrombogenic, clinically acceptable polymer (synthetic or natural) is applied to a damaged tissue site via minimally invasive techniques (e.g., via direct injection or via catheter). The polymer adheres to the target site and presents ligands for cell surface receptors expressed preferentially by the target stem/progenitor cells. It may also present moieties that induce cell proliferation and differentation, including sites that bind extracellular matrix or growth factors. Ligands may include peptide or carbohydrate sequences recognized by integrin or cadherin adhesion receptors and ligands for growth factor receptors such as FLK-1 or EGFR (erbB1). Selective capture of circulating cells does not depend on targeting a single, unique receptor (although that mode is not excluded), but can be accomplished by presenting a single ligand or multiple ligands in a manner that exploits avidity effects that intensify small differences in the total number of receptors expressed by different types of circulating cells.

For a single ligand, avidity effects may be exploited by presenting a fixed total amount of ligand in a nanoclustered rather than random format. A quantitative, nonlinear relationship exists between the density and spatial configuration of ligand in the cell environment, the number of receptors for the ligand expressed by the cell, and the cell adhesion strength. Adhesion is often observed as a threshold phenomena, so small quantitative differences in the total receptor expression between two cell types may lead to large differences in the cell adhesion strength to a particular ligand. Thus, two types of cells that express the same receptor but at slightly different levels might be differentially captured by a surface presenting a ligand for that receptor, if the ligand is presented in a way that magnifies the receptor differences between the cells. These avidity effects are more pronounced when two or more ligands for different receptors are presented by the polymer.

Instead of ligands for cell surface receptors, antibodies directed against cell surface antigens may also be used to effect selective cell capture. This approach may be preferred if selective ligands cannot be identified for the target cell, or if ligation of the target receptor induces a signaling process that results in adverse cell behavior (e.g., some growth factor receptors, such as EGFR, send an anti-adhesive signal acutely after ligation in some cell types).

Appropriate ligands may be identified via rational selection via known expression of cell surface receptors on the target cells, or ligands may be identified through a screening process against libraries of oligopeptides and oligosaccharides. A three-step screen is needed to ensure positive adhesion of the target cells, selection of target cells out of a population of blood cells, and non-thrombogenicity.

Ligand presentation at the target site may be accomplished via a number of different strategies. For example, ligand can be covalently linked to a comb copolymer with a relatively hydrophobic backbone (e.g., polymethylmethacrylate “PMMA”, and polylactic acid “PLA”) and multiple short, hydrophilic teeth (e.g., polyethyleneoxide “PEO”, and dextran oligomers) that serve as ligand tethers. Such polymers can be induced to gel upon warming, and a thin layer of such material could be formed in situ by injecting cooled polymer solution. The polymer may also be water-soluble, and attached to the tissue at the site via chemical or enzymatic crosslinking (e.g., using Factor XIIIa). The matrix may also be a porous or solid sheet-like device that is attached to the surface of the tissue locally via chemical or enzymatic crosslinking. Ligands may be attached with either a permanent covalent bond or with a hydrolyzable or enzymatically cleavable bond in order to create a locally tissue-responsive matrix, that enables a progression of signals to control cell differentiation after adhesion.

The polymer may also form a gel or solid that attaches to the tissue via chemical or enzymatic crosslinking and provides a matrix for cells to colonize the site. In addition to selecting cells, the matrix may contain additional molecules that induce growth, migration, or differentiation of the target cells. These factors may be covalently linked to the matrix, or may be dissolved in or reversibly bound to the matrix. To keep the blood-contacting surface non-thrombogenic, a gel-like matrix may be applied in a two-step process, with the outer surface containing lower densities moieties that induce migration and proliferation.

The matrix may also be applied in a way that provides structural guidance to regeneration and repair of a local site, and in this case again may be a composite with bulk properties that differ from the surface properties in chemical composition. One example would be coating a vascular stent with a non-thrombogenic polymer that selects hemangioblasts and angioblasts on the blood-contacting side and with a polymer that induces migration and differentiation of these cells on the vessel-contacting side. The polymer may also be delivered in a way that enables additional non-invasive characterization of the dynamics of healing at the site, such as inclusion of MRI contrast agents.

The polymer can be in the form of a matrix formed of fibrous scaffolding, for example, as described in U.S. Pat. No. 5,759,830 to Vacanti, et al. The design and construction of the scaffolding is of primary importance. The matrix should be a pliable, non-toxic, porous template for vascular ingrowth. The pores should allow vascular ingrowth and the injection of cells into the scaffold without damage to the cells or patient. The scaffolds are generally characterized by interstitial spacing or interconnected pores in the range of at least between approximately 100 and 300 microns in diameter. The matrix should be shaped to maximize surface area, to allow adequate diffusion of nutrients and growth factors to the cells and to allow the ingrowth of new blood vessels and connective tissue.

The same type of polymers can be used as in the Solid Free Form Manufacturing techniques described above. In the preferred embodiment, the matrix is formed of a bioabsorbable, or biodegradable, synthetic polymer such as a polyanhydride, polyorthoester, polyhydroxy acid such as polylactic acid, polyglycolic acid, or a natural polymer like polyalkanoates such as polyhydroxybutyrate and copolymers or blends thereof. Proteins such as collagen can be used, but is not as controllable and is not preferred. These materials are all commercially available. Non-biodegradable polymers, including polymethacrylate and silicon polymers, can be used, depending on the ultimate disposition of the growing cells.

In some embodiments, attachment of the cells to the polymer is enhanced by coating the polymers with compounds such as basement membrane components, agar, agarose, gelatin, gum arabic, collagens types I, II, III, IV, and V, fibronectin, laminin, glycosaminoglycans, mixtures thereof, and other materials, especially attachment peptides and polymers having attachment peptides or other cell surface ligands bound thereto, known to those skilled in the art of cell culture. Vitrogen—100 collagen (PCO 701) has been used in these experiments.

Polymeric materials which are capable of forming a hydrogel can also be utilized. The polymer is mixed with cells for implantation into the body and is permitted to crosslink to form a hydrogel matrix containing the cells either before or after implantation in the body. In one embodiment, the polymer forms a hydrogel within the body upon contact with a crosslinking agent. A hydrogel is defined as a substance formed when an organic polymer (natural or synthetic) is crosslinked via covalent, ionic, or hydrogen bonds to create a three-dimensional open-lattice structure which entraps water molecules to form a gel. Naturally occurring and synthetic hydrogel forming polymers, polymer mixtures and copolymers may be utilized as hydrogel precursors. See for example, U.S. Pat. No. 5,709,854 and WO 94/25080 by Reprogenesis.

In one embodiment, calcium alginate and certain other polymers that can form ionic hydrogels which are malleable. For example, a hydrogel can be produced by cross-linking the anionic salt of alginic acid, a carbohydrate polymer isolated from seaweed, with calcium cations, whose strength increases with either increasing concentrations of calcium ions or alginate. The alginate solution is mixed with the cells to be implanted to form an alginate suspension which is injected directly into a patient prior to hardening of the suspension. The suspension then hardens over a short period of time due to the presence in vivo of physiological concentrations of calcium ions. Modified alginate derivatives, for example, more rapidly degradable or which are derivatized with hydrophobic, water-labile chains, e.g., oligomers of ε-caprolactone, may be synthesized which have an improved ability to form hydrogels. Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Additional examples of materials which can be used to form a hydrogel include polyphosphazines and polyacrylates, which are crosslinked ionically, or block copolymers such as Pluronics™ or Tetronics™, polyethylene oxide-polypropylene glycol block copolymers which are crosslinked by temperature or pH, respectively. Other materials include proteins such as fibrin (although this is not preferred since thrombin may stimulate tumor growth via a pathway that MIS may have to overcome, such as EGF-stimulated proliferation), polymers such as polyvinylpyrrolidone, hyaluronic acid and collagen. Polymers such as polysaccharides that are very viscous liquids or are thixotropic, and form a gel over time by the slow evolution of structure, are also useful. For example, hyaluronic acid, which forms an injectable gel with a consistency like a hair gel, may be utilized. Modified hyaluronic acid derivatives are particularly useful. Polymer mixtures also may be utilized. For example, a mixture of polyethylene oxide and polyacrylic acid which gels by hydrogen bonding upon mixing may be utilized. In one embodiment, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

Covalently crosslinkable hydrogel precursors also are useful. For example, a water soluble polyamine, such as chitosan, can be cross-linked with a water soluble diisothiocyanate, such as polyethylene glycol diisothiocyanate. The isothiocyanates will react with the amines to form a chemically crosslinked gel. Aldehyde reactions with amines, e.g., with polyethylene glycol dialdehyde also may be utilized. A hydroxylated water soluble polymer also may be utilized.

Alternatively, polymers may be utilized which include substituents which are crosslinked by a radical reaction upon contact with a radical initiator. For example, polymers including ethylenically unsaturated groups which can be photochemically crosslinked may be utilized, as disclosed in WO 93/17669. Additionally, water soluble polymers which include cinnamoyl groups which may be photochemically crosslinked may be utilized, as disclosed in Matsuda et al., ASAID Trans., 38:154-157 (1992).

Standard immobilization chemistries, which are well known in the art, can be used to covalently link the tethers to the ligand and the matrix. Tethering ligands can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is glutaraldehyde. These and other attachment agents, as well as methods for their use in attachment, are described in “Protein immobilization: fundamentals and applications” Richard F. Taylor, ed. (M. Dekker, New York, 1991). Ligands can be tethered to a matrix by chemically cross-linking a tether molecule to reactive side groups present within the matrix and to a free amino group on the ligand. For example, synthetic EGF may be chemically cross-linked to a matrix that contains free amino or carboxyl groups using glutaraldehyde or carbodiimides as cross-linker agents. In this method, aqueous solutions containing free tethers molecules are incubated with the matrix in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art and can be used to join matrixs, tethers, and ligands.

For the disclosed cell growth compositions, ligands may be tethered either alone or in combinations. For example, both insulin and EGF may be tethered to the same matrix. The ligands may be combined in any desired proportions. The relative amounts of different ligands can be controlled, for example, by first separately linking the ligands to tethers, then mixing the “loaded” tethers in the desired proportions and attaching them to the matrix. The proportion of each ligand tethered to the substrate should match the proportion of loaded tethers in the attachment reaction.

Known methods and compositions for culturing cells and implanting them into the body can be adapted to use tethered ligands. For example, U.S. Pat. No. 4,352,883 to Lim, uses cells that are encapsulated within alginate microspheres, then implanted. Such microspheres can be modified with tethered ligands to improve their usefulness. Culturing cells on a matrix for use as artificial skin, as described by Yannas and Bell in a series of publications, can also be modified by tethering ligands to the matrix. U.S. Pat. No. 4,485,097 to Bell, U.S. Pat. No. 4,060,081 to Yannas et al., and U.S. Pat. No. 4,458,678 to Yannas et al. describe substrates for use as artificial skin. U.S. Pat. No. 4,520,821 to Schmidt describes a similar approach that was used to make linings to repair defects in the urinary tract.

Vacanti et al., Arch. Surg. 123: 545-549 (1988), describes a method of culturing dissociated cells on biocompatible, biodegradable matrices for subsequent implantation into the body. Cima and Langer, “Tissue Engineering” Chem. Eng. Prog. 89: 46-54 (1993), describe important considerations for the nature and form of implanted matrices useful for inducing tissue replacement. U.S. patent application Ser. No. 08/200,636 entitled “Tissue Regeneration Matrices by Solid Free Force Fabrication” filed Feb. 23, 1994 by Cima and Cima, which is hereby incorporated by reference, describes tissue regeneration matrices, fabrication techniques, and methods of regenerating tissue. In general, tissue regeneration devices can be constructed from polymers, ceramics, or from composites of ceramics and polymers. Common materials useful for constructing tissue regeneration devices are, for example, extracellular matrix proteins, especially collagens; proteins especially fibrin; degradable polyesters, such as polylactic acid, polyglycolic acid, co-polymers of polylactic acid and polyglycolic acid, and polycapralactone; polyhydroxybutyrate; polyanhydrides; polyphosphazenes; bone powder; natural polysaccharides, such as hyaluronic acid, starch, and alginate; hydroxyapatite; polyurethanes; and other degradable polymers described by Engleberg and Kohn, Biomaterials 12: 292-304 (1991). All of these known compositions can be modified by tethering ligands to the matrix.

Ligand tethered compositions for in vivo use can be in the form of polymeric, attachment molecule-coated sutures, pins, wound dressings, fabric, and space-filling materials. Attachment matrixs that promote ingrowth of dermal fibroblasts and capillaries could also be used for dermatological applications and cosmetic surgery, such as repair of wrinkles and aging skin, burn therapy, or skin reconstruction following disfiguring surgery. Matrixs with tethered ligands that promote osteoblast migration could be used to fill bone defects following tumor surgery or for non-healing fractures. Matrixs with tethered ligands that promote muscle cell growth and migration could be used for replacement of muscle mass, including cardiac muscle and smooth muscle, following disfiguring surgery and for patients with muscle degeneration or dysfunction. Tubular matrixs with tethered ligands that promote growth, migration, and function of epithelial, endothelial and mesenchymal cells can be used for construction of artificial ducts for carrying bile, urine, gases, food, semen, cerebrospinal fluid, lymph, or blood. Sheaths formed of matrixs that promote growth of fibroblasts from perichondrium, periosteum, dura mater, and nerve sheaths may be used to recreate these structures when they are injured or lost due to surgery or cancer. In all of these embodiments, either the matrix with tethered ligands or matrix plus attached cells may be used for reconstruction in vivo.

Matrixs for promoting tissue generation can be formed to have a desired tissue shape. As used herein, a desired tissue shape is the shape that the newly generated tissue is desired to have. For example, certain tissues may need to be sheet-like, tubular, or formed as a lobe.

### EXAMPLE 1

### Endogenous Bone Marrow Cells Traffic to the Heart and Differentiate into Cardiomyocytes Following Myocardial Infarction

Myocardial infarction results in the loss of cardiomyocytes, with subsequent scarring and impaired contractility. There has been substantial recent interest in repopulating the infarcted heart with cells introduced by intravenous or intramyocardial injection (see Kessler, P. D. & Byrne, B. J. Annu Rev Physiol 61, 219-242 (1999); Rosenthal, N. N Engl J Med 344, 1785-1787 (2001); Rosenthal, N. & Tsao, L. Nat Med 7, 412-413. (2001); Springer, et al. J Clin Invest 107, 1355-1356. (2001)). Many cell types including skeletal myoblasts (Taylor, D. A. et al. Nat Med 4, 929-933. (1998)), fetal cardiomyocytes (Sakai, T. et al. J Thorac Cardiovasc Surg 118, 715-724. (1999)), bone marrow cells (Tomita, S. et al. Circulation 100, II247-256. (1999); Liechty, K. W. et al. Nat Med 6, 1282-1286. (2000); Orlic, D. et al. Nature 410, 701-705. (2001)), and liver stem cells (Malouf, N. N. et al. Am J Pathol 158, 1929-1935. (2001)) have shown some degree of incorporation into the infarct zone. It is not clear, however, if part of the normal response to infarction includes repopulation from afar, and as importantly, whether newly resident cells transdifferentiate into cardiomyocytes. Here, we replaced bone marrow in mice with that from transgenic mice carrying the β-galactosidase gene under the α-myosin heavy chain (αMHC) promoter, and then subjected the mice to permanent ligation of the left anterior descending artery. Beginning at two weeks after myocardial infarction, small populations of transplanted cells are found in the heart. Over time, the number of donor-derived cardiomyocytes increases, as does the number of cells in each presumptive clone. These cells express β-galactosidase, indicating cardiac gene expression, as well as actinin and titin in patterns that indicate they are cardiomyocytes. These studies demonstrate that endogenous bone marrow cells normally traffic to injured myocardium, transdifferentiate into cardiomyocytes, and can persist there and grow over time.

Bone marrow transplantation with cells carrying the β-galactosidase gene was used to follow their fate following bone marrow transplantation. The transplantation protocol involves lethal irradiation and results in over 95% replacement of the marrow with donor cells, as determined by FACS analysis of peripheral blood cells from Ly5.1 recipient mice transplanted with Ly5.2 bone marrow.

In initial experiments, lethally irradiated wild-type mice were reconstituted with bone marrow from transgenic gt-rosa mice (Zambrowicz, B. P. et al. Proc Natl Acad Sci USA 94, 3789-3794 (1997)), which ubiquitously express β-galactosidase. The mice were allowed to recover for one month, and were subjected to myocardial infarction by permanent ligation of the left anterior descending artery (LAD). Two days following infarction, no cells in the heart stained blue. Eight weeks following myocardial infarction, cells within the infarct zone expressed β-galactosidase, as detected by staining with the chromogenic substrate X-gal. Interestingly, outside the infarct zone, occasional individual cells stained blue as well. The staining was punctate, and the pattern was the same as that found in the heart tissue of the gt-rosa mice themselves. Similar results have recently been reported by Jackson, K. A. et al. J Clin Invest 107, 1395-1402. (2001), using purified SP cells from gt-rosa mice. These results demonstrate that bone marrow-derived cells traffic to the heart and are found in the infarct zone.

In many tissues, injury results in activation of committed progenitor cells or stem cells, or proliferation of differentiated cells. Skeletal muscle contains myoblasts and satellite cells that can serve as progenitor cells. It has long been held that the heart cannot regenerate, because of the lack of progenitor cells in the heart and the inability of heart muscle cells to divide. However, recent studies challenge this tenet. Bone marrow cells, embryonic stem cells, and other cell types have been introduced into the injured heart (Kessler, (1999); Taylor, et al. (1998); Sakai, T. et al. (1999); Tomita, S. et al. (1999); Liechty, K. W. et al. (2000); Orlic, D. et al. (2001); Malouf, N. N. et al. (2001); Kocher, A. A. et al. Nat Med 7, 430-436. (2001); Menasche, P. et al. Lancet 357, 279-280. (2001); Li, R. K. et al. J Thorac Cardiovasc Surg 119, 62-68. (2000); Scorsin, M. et al. J Thorac Cardiovasc Surg 119, 1169-1175. (2000)), and furthermore, there is evidence that resident cells in the heart may be able to divide (Beltrami, A. P. et al. N Engl J Med 344, 1750-1757. (2001)).

In this context, these results demonstrate that bone marrow cells spontaneously circulate, traffic to the injured myocardium, and transdifferentiate into cardiomyocytes following MI. The present study differs from those in which cells are injected intravenously or directly into the infarct zone, because this is a naturally occurring process.

Methods

Bone Marrow Transplantation

gt-rosa mice, wild-type C57BL/6 mice, and wild-type FVB mice were obtained from Jackson Laboratories. αMHC-lacZ mice were kindly provided by Dr. Loren Field. Bone marrow was harvested from the femur and tibia of donor mice and placed into ice-cold Media 199 supplemented with 10 mM HEPES pH 7.4. Recipient mice were treated with acidified water and kept under germ-free conditions for at least two weeks prior to transplantation. They were lethally irradiated using a cesium source (1050 rads total) while in a microisolater. Three hours later, each mouse received 10⁷ donor bone marrow cells injected into the tail vein in a volume of 0.5 ml.

Myocardial Infarction Model

Animals were anesthetized with ketamine and xylazine, intubated, and ventilated. A left anterior thoracotomy was performed, and the left anterior descending artery visualized. The artery was ligated with a 6-0 suture and the wound closed.

Histochemistry and X-gal Staining

Mice were euthanized and the heart dissected free from surrounding tissue. The area of infarct was photographed, and the heart was cut along a short axis through the infarct. Half of the tissue was processed for paraffin embedding, and half was frozen in OCT on dry ice. 5 micron sections were taken.

For β-galactosidase staining using X-gal, sections were fixed for 5 minutes with 0.5% glutaraldehyde, and incubated with 0.05% X-gal in 0.1 M sodium phosphate, pH 7.5, 10 mM KCl, 1 mM MgCl₂, 3 mM K₄Fe(CN)₆, 3 mM K₃Fe(CN)₆, and 0.1% Triton X-100 at 37° C. overnight.

Immunohistochemistry was performed using standard techniques. Sections were embedded in LR hard resin for electron microscopy.

Results

The study was conducted to determine whether the bone marrow-derived cells are cardiomyocytes, as opposed, for example, inflammatory cells or fibroblasts, using donor cells that all stain blue using X-gal. Bone marrow from transgenic mice carrying the β-galactosidase gene under the αMHC promoter (αMHC-lacZ mice) (Soonpaa, M. H. & Field, L. J. Am J Physiol 272, H220-226. (1997)) was used. The cardiomyocytes of these animals express β-galactosidase, but other cell types, including bone marrow cells, do not. Lethally irradiated wild-type mice were reconstituted with bone marrow from the αMHC-lacZ mice. After four weeks of recovery, the mice were subjected to LAD ligation. Two days following MI, no blue cells were found anywhere in the heart. At two weeks, four weeks, six weeks, and eight weeks, sections through the heart reveal expression of β-galactosidase in cells only within the infarct zone. No blue cells were found anywhere else in the heart, and no blue cells were noted in the transplanted animals without MI. Because the donor bone marrow came from the αMHC-lacZ transgenic mouse, donor cells would not express β-galactosidase unless and until they differentiate into cardiomyocytes and express αMHC. The staining pattern in these animals differed from that of the recipients of the gt-rosa mice, in that the staining was more uniform and resembled that found in the αMHC-lacZ mice themselves. Unlike the recipients of the gt-rosa marrow, the recipients of the αMHC-lacZ marrow showed no blue cells outside the infarct zone.

The number of donor-derived cells increased over time after infarction, appearing to plateau between 4 and 6 weeks following infarction. Some X-gal staining was found in clusters, which suggested that a single donor cell may have divided in situ. The maximum number of blue cells in αMHC recipients was lower at 8 weeks than observed in gt-rosa recipients.

To confirm the αMHC expression indeed was indicative of full cardiomyocyte differentiation, immunofluorescence staining was seen for alpha-actinin and titin. Blue cells express alpha-actinin and titin and stain for Z-bands and myofibrils typical of cardiomyocytes.

TABLE 1
X-gal positive cells following bone marrow transplantation
and myocardial infarction.

| Time after MI | n | # with MI | # X-gal + in MI | Mean # cells/MI |
|---|---|---|---|---|
| αMHC-lacZ | | | | |
| 2 days | 3 | 3/3 | 0/3 | 0 |
| 2 weeks | 8 | 8/8 | 8/8 | 16 |
| 4 weeks | 7 | 6/7 | 6/6 | 40 |
| 6-8 weeks | 9 | 6/9 | 6/6 | 38 |
| gt-rosa | | | | |
| 7 weeks | 4 | 3/3 | 3/3 | 99 |

These results demonstrate that it is possible to recruit and capture stem cells or progenitor cells at a site in need thereof. Capture can be achieved efficiently using immobilized ligands, at the site, to capture and retain the cells.

1. A method for tissue engineering comprising administering at a site in need thereof a ligand for stem cells or progenitor cells, wherein circulating stem cells or progenitor cells are captured by the ligand.

2. The method of claim 1 wherein the ligand is immobilized to a matrix material at the site.

3. The method of claim 1 wherein the ligand is incorporated into and released by a matrix material or depo at the site.

4. The method of claim 1 wherein the ligand is released from genetically engineered cells at the site.

5. The method of claim 1 wherein the ligand specifically binds to stem cells or progenitor cells.

6. The method of claim 1 further comprising providing molecules inducing proliferation or differentiation of stem cells or progenitor cells.

7. The method of claim 1 wherein the matrix is formed of a natural or synthetic polymer.

8. The method of claim 7 wherein the matrix is a fibrous mesh.

9. The method of claim 7 wherein the ligands are tethered to the matrix.

10. A matrix for use in a method for tissue engineering comprising administering at a site in need thereof a ligand for stem cells or progenitor cells, wherein circulating stem cells or progenitor cells are captured by the ligand.

11. A genetically engineered cell for use in a method for tissue engineering comprising administering at a site in need thereof a ligand for stem cells or progenitor cells, wherein circulating stem cells or progenitor cells are captured by the ligand.
